(19) United States
(12) Patent Application Publication
(10) Pub. No.: US 2004/0260285 A1
(43) Pub. Date: Dec. 23, 2004

(54) Pedicular hooks for rachidian anchoring device

(75) Inventors: Jean-Paul Steib, Strasbourg (FR); Jean-Yves Leroy, Campagne Les Hesdin (FR); Rodolphe Lourdel, Beuvry (FR); Pascal Rokegem, St. Laurent Blangy (FR); Guy Viart, Saint Leger (FR)

Correspondence Address: YOUNG & THOMPSON, 745 SOUTH 23RD STREET, 2ND FLOOR, ARLINGTON, VA, 22202, US

(73) Assignee: EUROSURGICAL SA, Beaurains (FR)

(21) Appl. No.: 10/690,585

(22) Filed: Oct. 23, 2003

(30) Foreign Application Priority Data
Jun. 17, 2003 (FR) 03 07253

(51) Int. Cl. A61B 17/58

(52) U.S. Cl. 606/61

(57) ABSTRACT

The rachidian anchoring device with pedicular hooks (2, 4), permitting the emplacement and securement of a connecting rod (3) at the level of each vertebra (9) of a vertebral column, comprises particular hooks (2, 4) provided with securement means for the reception and blocking of a transverse rod (5) connecting said pedicular hooks (2, 4) together and permitting exerting compression between the two pedicular hooks (2, 4) in the direction of the center of the vertebra (9).

The present invention relates to a pair of pedicular hooks for a rachidian anchoring device and more particularly for guidance and holding of the connecting rods of said device at the level of each vertebra of the vertebral column.

The rachidian anchoring device according to the present invention is fixed on each vertebra of a rachidian segment to be acted on of a vertebral column after resection of the transverse pedicles and of the epineural.

The rachidian anchoring device according to the present invention is retained on each vertebra by compression of the two pedicular hooks in the direction of the center of said vertebra. The pedicular hooks bear respectively on the base of the pedicles to ensure good retention of the rachidian anchoring device.

The rachidian anchoring device with pedicular hooks permitting the emplacement and securement of a connecting rod at the level of each vertebra of a vertebral column, comprises pedicular hooks provided with securement means for the reception and blocking of a transverse rod connecting said pedicular hooks together and permitting a compression of the two pedicular hooks in the direction of the center of the vertebra.

The rachidian anchoring device according to the present invention comprises a first pedicular hook comprising an osseous anchoring and an open body for the reception and securement of the connecting rod by means of a clip by means of a pressure screw, said open body comprising on its external surface a prolongation provided with securement means for the reception and blocking of the transverse rod and a second pedicular hook comprising an osseous anchoring portion and an open body provided with securement means for the reception and securement of the transverse rod.

The description which follows with respect to the accompanying drawings, given by way of non-limiting examples, will permit better understanding the invention, the characteristics that it has and the advantages which it is adapted to provide:

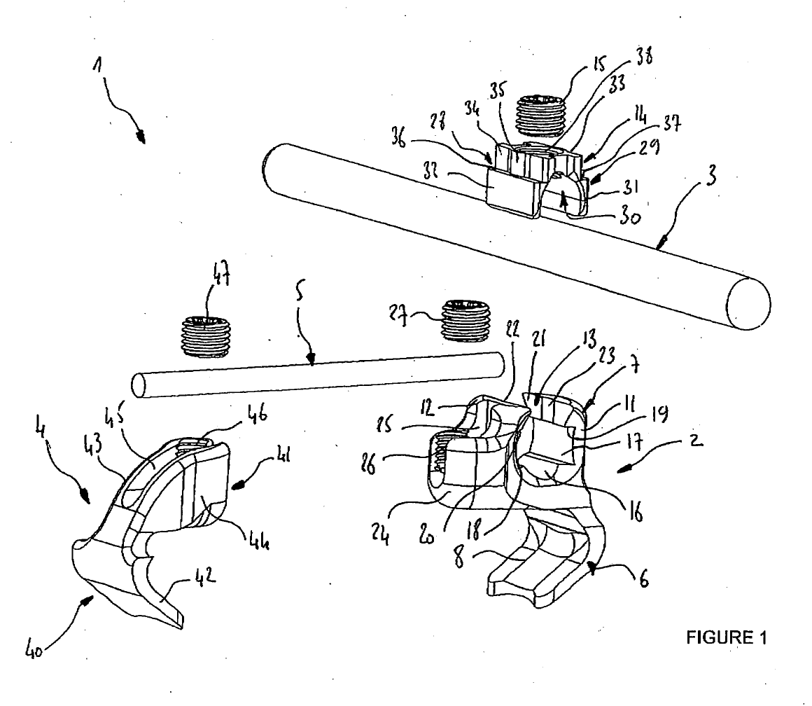

FIG. 1 is an exploded perspective view showing a rachidian anchoring device with pedicular hooks according to the present invention.

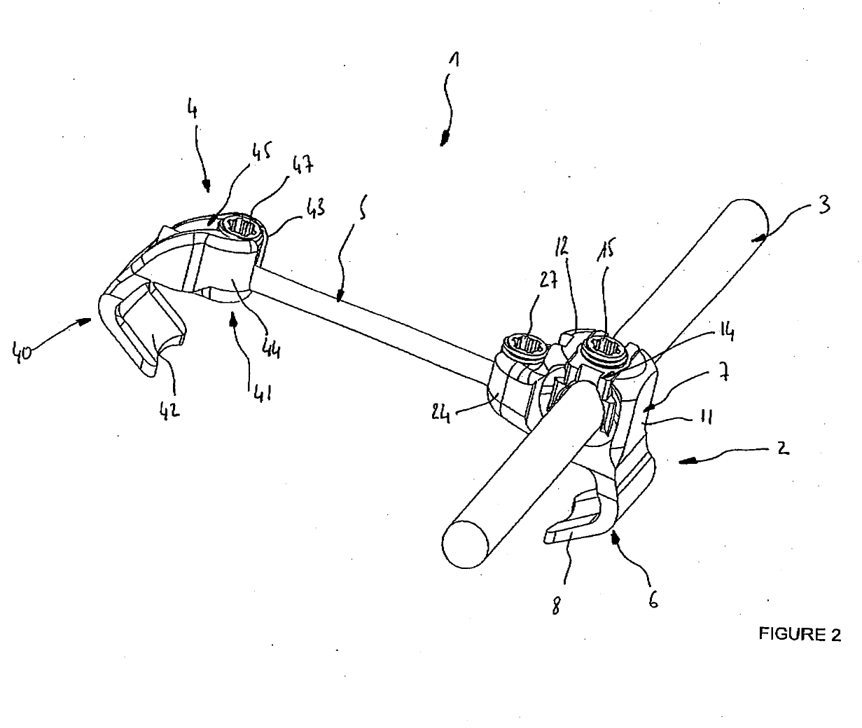

FIG. 2 is a perspective view showing the rachidian anchoring device with pedicular hooks in the assembled position according to the present invention.

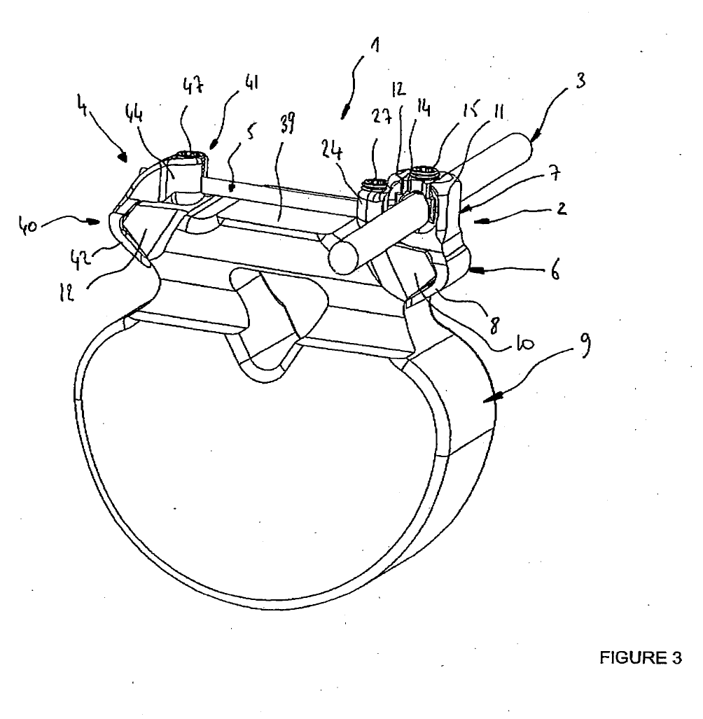

FIG. 3 is a perspective view showing the rachidian anchoring device with pedicular hooks in a position mounted on vertebral bodies of a vertebral column.

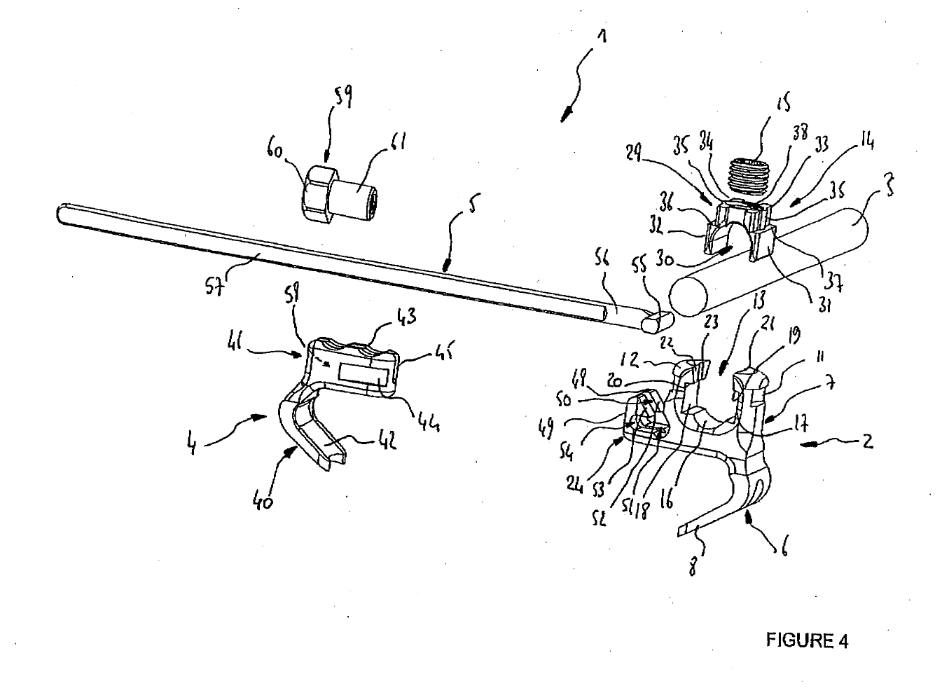

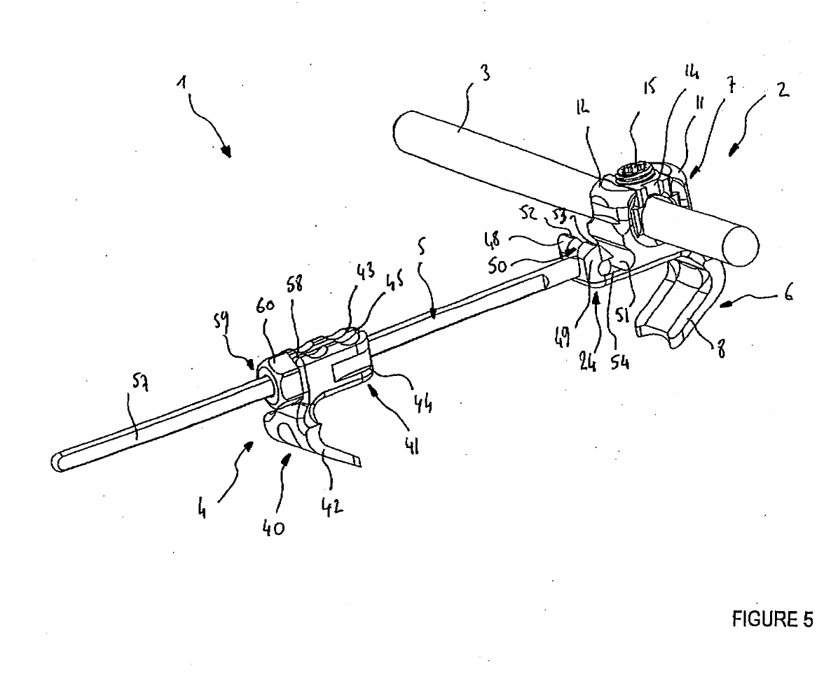

FIGS. 4 and 5 are perspective views showing a first modification of the rachidian anchoring device with pedicular hooks according to the present invention.

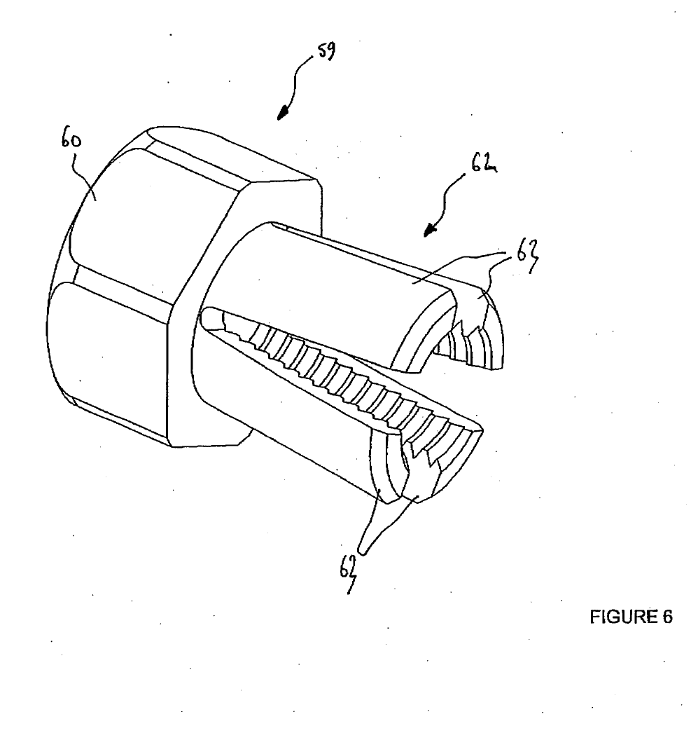

FIG. 6 is a perspective view showing a modification of a locking nut of the rachidian anchoring device with pedicular hooks according to the present invention.

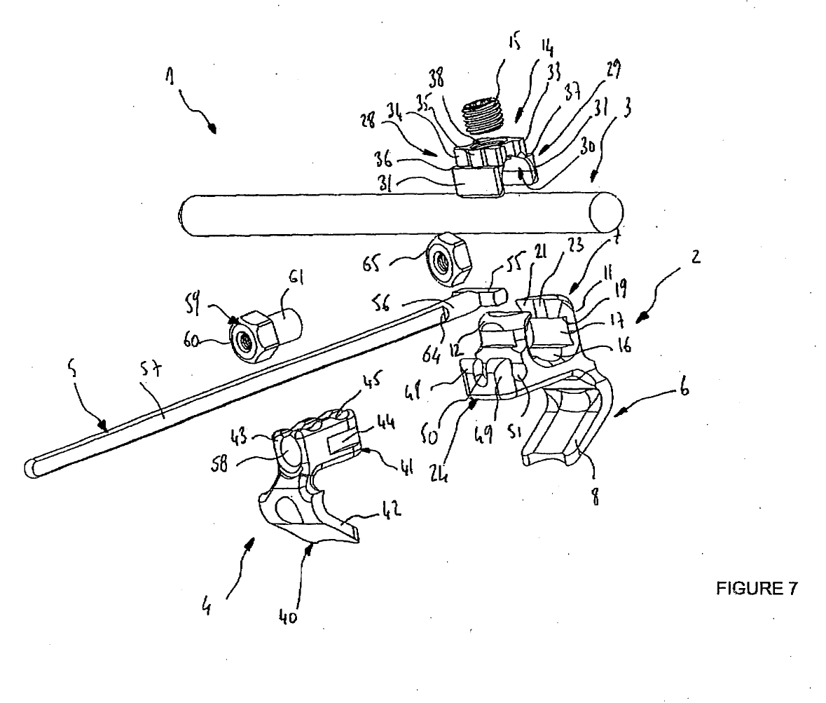

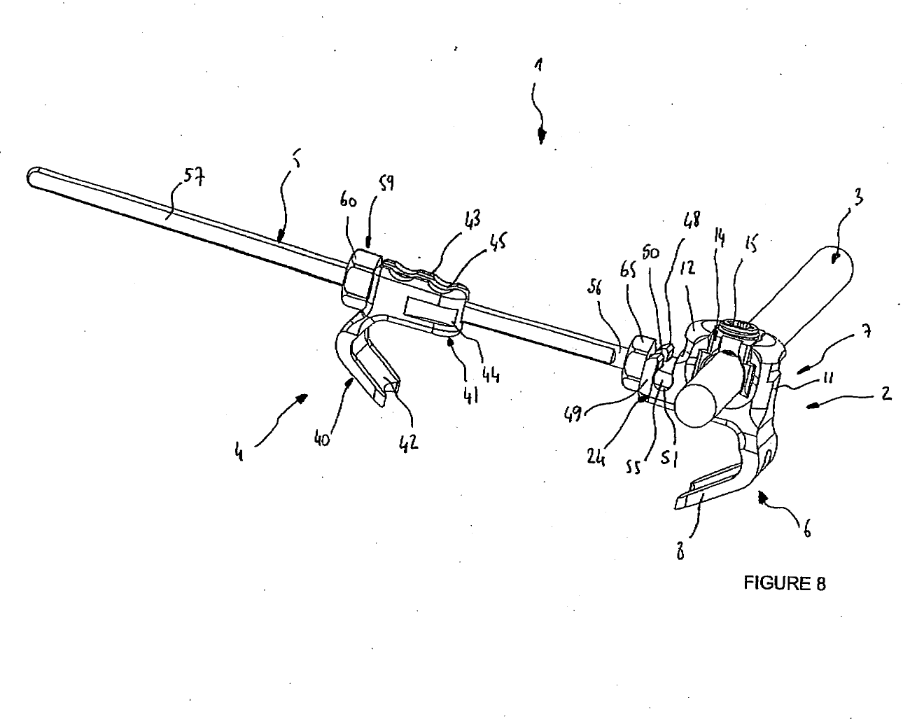

FIGS. 7 and 8 are perspective views showing another modification of the rachidian anchoring device with pedicular hooks according to the present invention.

There is shown in FIG. 1 to 3 a rachidian anchoring device 1 comprising a first pedicular hook 2 adapted to receive a connecting rod 3 and a second pedicular hook 4 connected to the first by a tranverse rod 5 having a smooth cuttable cylindrical profile.

The first pedicular hook 2 comprises an osseous anchoring portion 6 prolonged by an open body 7 for the reception and securement of the connecting rod 3.

The osseous anchoring portion 6 is constituted by a blade 8 in the form of a hook extending below the body 7 to hook and secure on a vertebra 9 of a vertebral column and more particularly on the transverse pedicle 10 after resection.

The blade 8 in the form of a hook is disposed below the open body 7 in a same vertical plane such that the hooking zone on the transverse pedicle 10 will be substantially perpendicular to the direction of the connecting rod 3.

The open body 7 of the first pedicular hook 2 comprises two vertical and lateral branches 11, 12 delimiting an opening 13 of U shape, provided to receive a clip 14 provided with a pressure screw 15 for blocking, in the bottom of the opening 13, the connecting rod 3.

The vertical branches 11 and 12 comprise, on their internal surfaces and above the bottom 16 of the opening 13, opposite grooves 17, 18 delimiting, at least in their upper portion, a flat shoulder surface 19, 20.

The vertical branches 11 and 12 comprise, in a vertical plane above and in a plane perpendicular to the shoulder surfaces 19, 20, flat surfaces 21, 22 each having a recess 23 in the form of the arc of a circle.

The vertical branch 12 of the open body 7 comprises on its external surface a prolongation 24 directed on the one hand perpendicularly to the direction of securement of the connecting rod 3 in the opening 13, and on the other hand above the blade 8 forming the hook.

The prolongation 24 comprises an elongated recess 25 of U shape opening outwardly of the prolongation and having in a vertical direction a screw-threaded portion 26 coacting with a pressure screw 27 for blocking the transverse rod 5 in said recess.

The clip 14, coacting laterally slidably with the opening 13 of the open body 7, comprises two vertical and lateral walls 28, 29 delimiting an opening 30 of U shape provided to receive the connecting rod 3.

The vertical walls 28, 29 comprise, on the one hand first flat surfaces 31, 32 adapted to coact respectively with the grooves 17, 18 and on the other hand second flat surfaces 33, 34 each having a prismatic projection 35 coacting with the recess 23 of the surfaces 21, 22 of the vertical branches 11 and 12.

The first flat surfaces 31, 32 and the second flat surfaces 33, 34 of the vertical walls 28, 29 are respectively offset from each other to constitute flat shoulders 36, 37 which each come into bearing against vat 19, 20 of the grooves 17, 18 upon gripping of the pressure screw 15 against the connecting rod 3.

Thus, the clip 14 comprises, in its upper portion and in a vertical direction, a screw-threaded bore 38 opening into the opening 30 and coacting with the pressure screw 15 to permit the securement and immobilization on the one hand of the clip 14 in the body 7 of the hook 2 and on the other hand of the connecting rod 3 in the hook 2.

The second pedicular hook 4 of the rachidian anchoring device 1 comprises an osseous anchoring portion 40 prolonged by an open body 41 for the reception and securement of the transverse rod 5.

The osseous anchoring portion 40 is constituted by a blade 42 in the form of a hook extending below the body 41 to hook and fix against a vertebra 9 of a vertebral column and more particularly on a transverse resectioned pedicle 12, located opposite the first 10 coacting with the hook 2.

The open body 41 of the second pedicular hook 4 comprises two vertical and lateral branches 43, 44 delimiting an opening 45 of U shape, comprising a screw-threaded portion 46 coacting with a pressure screw 47 for blocking in the bottom of the opening 45 of the transverse rod 5.

The rachidian anchoring device 1 is fixed on each vertebra 9 of the rachidian segment to be acted on of a vertebral column after resection of the transverse pedicles 10, 12 and the epineural 39.

The rachidian anchoring device 1 is retained on each vertebra 9 by compression between the two pedicular hooks 2, 4 in the direction of the center of said vertebra. The hooks 2 and 4 bear respectively on the base of the pedicles 10, 12 to ensure good retention of the rachidian anchoring device 1.

The securement and blocking of the transverse rod 5 in the elongated recess 25 of the prolongation 24 of the first pedicular hook 2 by the pressure screw 27 and in the opening 45 of the second pedicular hook 4 by the pressure screw 47 permits on the one hand maintaining the compression of the pedicular hooks 2, 4 in the direction of the center of each vertebra 9 and on the other hand retaining the rachidian anchoring device.

As soon as the hooks 2 and 4 are held on each vertebra 9, it is provided to place the connecting rod 3 in each opening 13 of the body 7 of each hook 2 and to slide each clip 14 along the rod 3 so as to bring them into snapping engagement between the branches 11 and 12 of said body 7. The blocking in rotation and translation of the connecting rod 3 within each hook 2 is obtained by clamping the pressure screw 15 which also permits retaining the clip 14 in the opening 13.

There is shown in FIGS. 4 and 5 a first modification of the rachidian anchoring device 1 according to the present invention.

This modification is different from that described previously in FIGS. 1 to 3 principally as to the profile of the prolongation 24, of the transverse rod 5 and of the second pedicular hook 4, given that the assembly of the other elements constituting the rachidian anchoring device 1 is identical.

Thus, the first pedicular hook 2 comprises on the external surface of its vertical branch 14 of the open body 7 a prolongation 24 directed on the one hand perpendicularly to the direction of securement of the connecting rod 3 in the opening 13 and on the other hand above the blade 8 forming a hook.

The prolongation 24 has a profile of hook shape constituted by two parallel blades 48, 49 separated by a vertical slot 50 opening into a recess 51. Each blade comprises an inclined surface 52, 53 forming the upper and external portion of the hooks and a hooking portion 54 communicating with the recess 51.

The transverse rod 5 comprises a hooking portion of T shape constituted by an axle 55 disposed perpendicularly to the principal axis of the rod. The transverse rod 5 has, adjacent the axle 55, parallel flats 56 which are prolonged by a cylindrical and screw-threaded profile 57.

The second pedicular hook 4 comprises an osseous anchoring portion 40 prolonged by an open body 41 for the reception and securement of the transverse rod 5.

The open body 41 is distinguished from that described previously in that the opening 45 of U shape, delimited by the vertical and lateral branches 43, 44, has, at one of its ends and in a horizontal direction, a cylindrical bore 58 permitting the reception and axial guidance of a locking nut 59.

The nut 59 has a gripping head 60 with a hexagonal profile or the like which is prolonged by a hollow cylinder 62 which is screw-threaded in its internal portion to coact with the screw-threaded profile 57 of the transverse rod 5. As a modification, the nut 59 can have in the prolongation of it gripping head 60, a hollow internally screw-threaded cylinder 62 cut longitudinally into several identical portions 63 (FIG. 6).

This modification permits providing a nut 59 with rapid locking permitting sliding freely on the screw-threaded cylindrical profile 57 of the transverse rod 5 up to the level of the open body 41 of the second pedicular hook 4.

Then, the portions 63 of the hollow cylinder 62 are again gripped to be able to penetrate by screwing into the interior of the bore 58 of the open body 41 of the second pedicular hook 4.

The assembly of the rachidian anchoring device 1 described in FIGS. 4 and 5 is similar to that described previously, namely, that it is secured on each vertebra 9 of a rachidian segment to be operated on of a vertebral column after resection of the transverse pedicals 10, 12 and of the epineural 39.

The rachidian anchoring device 1 is retained on each vertebra 9 by compression between the two pedicular hooks 2, 4 in the direction of the center of said vertebra. The hooks 2 and 4 bear respectively on the base of the pedicals 10, 12 to ensure good holding of the rachidian anchoring device 1.

Thus, the transverse rod 5 is introduced into the recess 51 of the prolongation 24 of the first pedicular hook 2 such that the axle 55 of T shape coacts with the hooking portion 54 of the blades 48, 49.

Upon mounting of the transverse rod 5 in the prolongation 24 of the first pedicular hook 2, it will be noted that the flats 56 coact with the vertical slot 50, provided between the blades 48, 49, so as to block in rotation about its longitudinal axis the transverse rod 5.

The transverse rod 5 coacts, opposite its axle 55, with the opening 45 of the open body 41 of the second pedicular hook 4. The compression of the rachidian anchoring device 1 is effected by the locking of the nut 59 on the transverse rod 5 until its hollow cylinder 61, 62 enters the interior of the bore 58 of the open body 41.

It will be noted that the locking nut 59 permits on the one hand varying the compression between the hooks 2 and 4 and on the other hand preventing any expulsion of the transverse rod 5 of the second hook 4 because of the coaction of the hollow cylinder 61, 62 with the bore 58.

There is shown in FIGS. 7 and 8 a second modification of the rachidian anchoring device 1 according to the present invention.

This modification is different from the one previously described in FIGS. 4 to 6, principally as to the profile of the prolongation 24 and of the transverse rod 5, given that the assembly of the other elements constituting the rachidian anchoring device 1 is identical.

Thus, the prolongation 24 is distinguished from that described in FIGS. 4 and 5 in that the vertical blades 48 and 49 no longer comprise inclined surfaces 52, 53 and anchoring portion 54. On the contrary, the vertical blades 48 and 49 are again separated by a vertical slot 50 for the passage of the transverse rod 5.

Similarly, the prolongation 24 comprises between the blades 48 and 49 and the external surface of the vertical branch 12 of the open body 7, a recess 51 whose dimensions are sufficient to receive the axle 55 of the transverse T shaped rod 5.

On the other hand, the transverse rod 5 comprises at the level of its flats 56 a screw-threaded portion 64 coacting with another locking nut 65 permitting locking the return of the rod to the level of its T shaped axle 55 relative to the first pedicular hook 2.

The assembly of the rachidian anchoring device is similar to that described in FIGS. 4 to 6 as to the compression between the pedicular hooks 2 and 4 against a vertebra 9 by means of the transverse rod 5.

The advantage of locking the return of the rod 5 at the level of its axle 55 can be applied to the modification described and shown in FIGS. 4 and 5.

It must further be understood that the description which has been given has been given only by way of example and that it in no way limits the scope of the invention, which will not be exceeded by replacing the details of execution described, by any other equivalent.

1. Rachidian anchoring device with pedicular hooks (2, 4), permitting the emplacement and securement of a connecting rod (3) at the level of each vertebra (9) of a vertebral column characterized in that each pedicular hook (2, 4) comprises securement means for the reception and blocking of a transverse rod (5) connecting said pedicular hooks (2, 4) together and permitting compression between the two pedicular hooks (2, 4) in a direction toward the center of the vertebra (9).

2. Rachidian anchoring device according to claim 1, characterized in that it comprises a first pedicular hook (2) comprising an osseous anchoring portion (6) and an open body (7) for the reception and securement of the connecting rod (3) by means of a clip (14) by means of a pressure screw (15), said open body (7) comprising on its external surface a prolongation (24) provided with securement means for the reception and blocking of the transverse rod (5) and a second pedicular hook (4) comprising an osseous anchoring portion (40) and an open body (41) provided with securement means for the reception and securement of the transverse rod (5).

3. Rachidian anchoring device according to claim 2, characterized in that the prolongation (24) is directed on the one hand perpendicularly to the direction of securement of the connecting rod (3) and on the other hand above the osseous anchoring portion (6) forming a hook.

4. Rachidian anchoring device according to claim 2, characterized in that the prolongation (24) comprises a recess (25) of U shape opening externally of the prolongation and having in a vertical direction a screw-threaded portion (26) coacting with a pressure screw (27) for the blocking of the transverse rod (5) in said recess.

5. Rachidian anchoring device according to claim 2, characterized in that the prolongation (24) comprises two parallel blades (48, 49) separated by a vertical slot (50) opening into a recess (51) for the reception of the transverse rod (5).

6. Rachidian anchoring device according to claim 5, characterized in that each parallel blade (48, 49) comprises an inclined surface (52, 53) forming the upper and external portion of a hook and a hooking portion (54) communicating with the recess (51) for the reception of the transverse rod (5).

7. Rachidian anchoring device according to claim 2, characterized in that the open body (41) of the second pedicular hook (4) comprises two vertical and lateral branches (43, 44) delimiting a U shaped opening (45) comprising a screw-threaded portion (46) coacting with a pressure screw (47) for the blocking in the bottom of the opening (45) of the transverse rod (5).

8. Rachidian anchoring device according to claim 2, characterized in that the open body (41) of the second pedicular hook (4) comprises two vertical and lateral branches (43, 44) delimiting a U shaped opening (45) comprising, at one of its ends and in a horizontal direction, a cylindrical bore (58) permitting the reception and axial guidance of a locking nut (59) for the blocking of the transverse rod (5).

9. Rachidian anchoring device according to claim 4, characterized in that the transverse rod (5) has a cuttable smooth cylindrical profile.

10. Rachidian anchoring device according to claim 5, characterized in that the transverse rod (5) comprises a T shaped hooking portion constituted by an axle (55), adjacent the axle (55) parallel flats (56) which are prolonged by a cylindrical and screw-threaded profile (57).

11. Rachidian anchoring device according to claim 10, characterized in that the transverse rod (5) comprises at the level of its flats (56) a screw-threaded portion (64) coacting with a locking nut (65) permitting locking the return of the rod to the level of its axle (55) relative to the first hook (2).

12. Rachidian anchoring device according to claim 8, characterized in that the nut (59) has a gripping head (60) which his prolonged by a hollow cylinder (61) which is screw-threaded in its internal portion to coact with the screw-threaded profile (57) of the transverse rod (5).

13. Rachidian anchoring device according to claim 8, characterized in that the nut (59) can have in the prolongation of its gripping head (60) a hollow cylinder (62) internally screw-threaded and longitudinally cut into several identical portions (63).
